(19) United States
(12) Patent Application Publication
(10) Pub. No.: US 2008/0003274 A1
(43) Pub. Date: Jan. 3, 2008

(54) Decompressive thermogenic bandage

(76) Inventor: Daniel E. Kaiser, Henderson, NV (US)

Correspondence Address: LAW OFFICE OF JAY R. HAMILTON, PLC., 331 W. 3RD ST., NEW VENTURES CENTER SUITE 120, DAVENPORT, IA, 52801, US

(21) Appl. No.: 11/477,174

(22) Filed: Jun. 28, 2006

(51) Int. Cl. A61K 9/70

(52) U.S. Cl. 424/449

(57) ABSTRACT

An apparatus is disclosed that allows for the delivery of medications and or chemicals for treatment of tissue through permeable dermal patch positioned inside a vessel for use in treating of any particular disease or aliment. The vessel self-adheres through activation of a chemical heater which may be activated by a pull tab exterior of the vessel. Initiation of the chemical reaction generates heat sufficient to raise the air temperature inside the vessel to allow the vessel to auto-generate a vacuum for wound treatment and to aid in sterilization of the wound site. The heated air expands and is evacuates through a one-way valve exterior of the vessel. Cooling of the interior air generates a vacuum inside the vessel to create a suction that holds the vessel in place. Increases blood flow to the treated tissue area, stimulated by the vacuum, facilitates improved delivery of the medication or pharmacological composition to the user. Application of vacuum to the treated tissue disinfects the tissue while providing a bacterial infiltration barrier.

### CROSS REFERENCE TO RELATED APPLICATIONS

(Not Applicable)

This non-provisional patent application claims priority from the provisional patent application previously filed on Jun. 28, 2005 and assigned Ser. No. 60,694,757 by the United States Patent & Trademark Office. This application seeks both United States and International protection for the inventions and inventive methods disclosed herein under both the laws of the United States and the agreed accords of the Paris Convention Treaty (PCT). Patent applications having the following titles and applicant attorney assigned docket numbers are filed concurrently in the United States Patent & Trademark Office and are incorporated by reference herein: USPA0200 “Apparatus for Vascular and Nerve Tissue Histogenesis and Enhancement”; USPA0205 “Method for Histogenesis and Enhancement of Tissue”; and, USPA0215 “Selective Destruction of Cancerous Cellular Tissue”.

### FIELD OF THE INVENTION

An auto-generating vacuum device (self-vacuumizing bandage) for wound treatment to aid in sterilization of wound site through bacterial destruction, improve medicament delivery and improve and enhance the tissue healing process through increased blood flow to the cells and cellular structure in and around the wound. This apparatus and structure allows a safe non-invasive method of tissue histogenesis for treatment of tissues and tissue structures on or near the surface of the body. As disclosed the present art, it increases the strength and mass of cell membranes, creates a favorable environment for the redevelopment of micro-vascular tissues and reduces the development of scare tissue.

### STATEMENT REGARDING FEDERALLY SPONSORED RESEARCH OR DEVELOPMENT

No federal funds were used to develop or create the described disclosure.

### REFERENCE TO SEQUENCE LISTING, A TABLE, OR A COMPUTER PROGRAM LISTING COMPACT DISK APPENDIX

(Not Applicable)

### BACKGROUND OF INVENTION

The normal animal cell, including that of humans, has in general a predefined shape and size. It has been discovered when sufficiently stressed, the cell will increase in size and its external structure will also deviate to accommodate any vacuum or negative force that is applied to the cell. Proper application of vacuum to the cellular structure can induce the cell to replicate and/or accommodate the stress that is applied by the vacuum. The resiliency of cellular membranes and its supporting structure, as noted in the prior art and as discovered in the use of this invention, can be damaged beyond repair by the improper application of an excessive amount of vacuum. Therefore, it is critical that the amount of vacuum be properly controlled and limited both manually and automatically to avoid damage to the tissues, including but not limited to their internal mechanisms and membranes.

This invention has shown that animal cellular structures can accommodate vacuums from 0.0009 inches of Hg to 30 inches of Hg. Improperly applied lower pressures and stresses if not used in accordance with this invention and its method of operation may also cause cellular damage. It is theorized, however, that if the body's tissues are stimulated properly and the methods applied in accordance within tissue limits with this invention, that forces and stresses even higher might safely be obtained. The body's immune system can routinely repair most, if not all, damage caused by minimal to medium amounts of vacuum in healthy tissues. This is similar to the repair of minor contusions, discoloration and vascular seepage caused by small amounts of vacuum such as that which can be applied to the skin by the vacuum induced by the mouth. The vacuum force (decompression) acts to cause the veins and arteries to enlarge and engorge providing the benefits of increased blood flow. The increase in blood flow, due to enlargement and or enhancement of healthy and normal blood vessels would benefit substantially by increasing the malleability, strength, and overall health of the vessels themselves. The increase in blood flow would, over time, improve the surrounding cells and provide more nutrients to damaged areas to aid in the repair of wounds and/or unhealthy tissue that lacked proper oxygen levels. It would appear to be useful on most any tissue that has morphemic characteristics.

### SUMMARY OF THE INVENTION

As disclosed, the present art is a novel technology and method for application within the medical technology as well as the biological technology fields. The disclosed concepts revolve around the application of decompressive vacuum or vacuum energy to different elements as well as form, function and homeostasis affecting the cellular biology, neurology, immunology and vascular tissues of humans and animals. This invention allows for the controlled development of increased blood flow near the surface of the human body. The method and apparatus disclosed and claimed herein allow the delivery of mechanical force in a safe and non-invasive way to the outer tissues of the body to stimulate the natural healing mechanisms and the body's ability to maintain a homeostasis state.

The technology has many other therapeutic uses including immune system enhancement, cellular development, vascular and neurological system development. This technology is efficacious in controlling the growth of infectious agents and organisms. As disclosed, the components of the technology include the design of the vessel, the application of the seal between the vessel and the tissue, the ignition and in tissue to be treated and the method of treatment of the tissue.

One facet of the vessel design is that it can be constructed of any transparent and or opaque material that is so engineered and or designed to withstand vacuum or negative pressure and or decompressive energy within said vessel to a value of up to 30 inches of mercury (Hg).

The device as designed can be made of many interlocking sealing segments and or come as a custom molded unit that is patient specific. Some applications will require customization of the vessel and others will not. The design of the vessel will be determined by the needs of the patient and or the specific treatment area and or the therapy necessary to stimulate the desired tissue response i.e. tissue growth, vascular regeneration, neural network regeneration, increased blood flow and pharmaceutical delivery.

The single most obvious benefit of this invention is the controlled development of increased blood flow inside the human body as well as an increase in micro vascularization throughout the treatment area. This mechanical force is delivered in a safe and non-invasive way as to stimulate the natural healing mechanisms of the body's ability to maintain a homeostasis state.

If this method and apparatus is used within a range of 1-9 inches of Hg, at the beginning of the hyper-enhancement process, small and superficial contusions or bruising may occur. It has been determined that the comfort level of vacuum should be gradually increased over a period of time, starting from approximately 1.0-1.5 inches of Hg and proceeding to higher values of vacuum and decompression.

This invention has also been utilized with variations in the configuration of the dome, sphere, or shape of a vacuum applicator and/or containment vessel. Varying the shape of the vacuum applicator varies the forces exerted upon and into the material or tissue exposed to vacuum energy. Thus, the tissue may be elongated, lengthened, or widened by enhancement or expansion within and in conjunction with the sphere.

This invention allows application of larger amounts of vacuum or negative pressure to be applied to specific tissues under substantial control to decompress tissue within a containing device or vessel without damaging surrounding supporting tissues for the enhancement of the tissue within the vessel.

A medical device and methods for the treatment of cuts, wounds, scrapes, post amputation wounds, diabetic ulcers and bedsores as well as other medical disorders and ailments that would benefit from increased and enhanced tissue response and wound healing due to increases in blood flow on both a macro and micro vascular level including increased cellular stimulation, improved delivery of pharmacological solutions and medicants other situations requiring elimination of active infections and stimulating wound healing response is disclosed and claimed herein.

The seal between the vacuum container and the human cells or tissues surrounding the tissues (tissues to be enhanced) permits the use of a dynamic vacuum force which will stimulate cell activity without permanent harm to cells and/or user.

It is therefore an attribute of the design as disclosed and claimed to allow the tissue to be affected by vacuum to be placed inside and or underneath the vacuum device.

It is another attribute of the design to allow for placement of the device on a body part to affect vacuum treatment.

It is another attribute of the vessel design to control the application of vacuum energy by contouring or shaping the vessel in such a way as to modulate the response of the tissue to the vacuum to affect the desired change in therapeutic application and to control stimulation rate, growth rate and or blood flows.

It is therefore an object of the present invention to provide a non-invasive method to stimulate and improve tissues.

It is still another object of this invention is to stimulate increased blood flow in tissue and vascular systems.

It is a further object of the invention to provide a system and method that allows for deep penetration of vacuum based into human or animal tissues.

It is still another object of this invention is to provide a method and technology that stimulates tissue and cellular growth.

It is still another object of this invention is to provide a method and technology that stimulates the strength, flexibility, and expandability of tissues and or cellular membranes and internals.

### BRIEF DESCRIPTION OF THE DRAWINGS

FIG. 1 is an isometric view of a self-adhering, vacuum based medicine delivery chamber that also helps to create a sterile zone within the chamber.

FIG. 2 is an illustration of the decompression gradient produced by application of decompression energy to tissue.

### DETAILED DESCRIPTION—ELEMENT LISTING

| | |
|---|---|
| ONE-WAY CHECK VALVE | 1 |
| ACTIVATION TAB | 2 |
| CHEMICAL HEAT GENERATOR | 3 |
| CASE BASE | 4 |
| VACUUM VESSEL | 5 |
| PERMEABLE MEDICATED DERMAL | 6 |
| PATCH (OR MEMBRANE) | |
| DECOMPRESSIVE ENERGY UNITS | 7 |
| SOFT AND OR PERMEABLE TISSUE | 8 |
| DECOMPRESSIVE ENERGY GRADIENT | 9 |
| ATMOSPHEREIC PRESSURE, DECOMPRESSSIVE | 10 |
| ENERGY IS NEUTRAL | |
| AUTO-GENERATING VACUUM BANDAGE | 14 |

### DETAILED DESCRIPTION

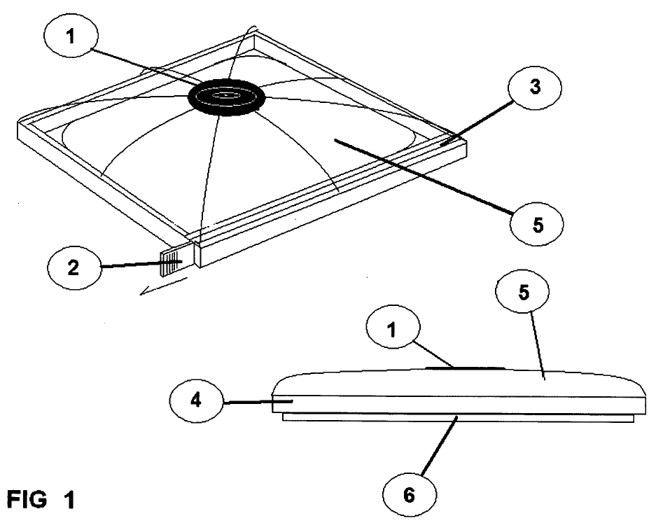

FIG. 1 is an embodiment of this invention that allows for the delivery of medications and or chemicals for treatment via a permeable dermal patch 6, which is housed inside the case base 4, for use in treating any particular disease or aliment. The self-adhering action of this device is activated by means of a chemical heat generator 3, which is activated by means of an activation tab 2. When the divider and activation tab 2 are removed, a mixture of chemicals cause a generation of heat sufficient enough to raise the air temperature inside the device. As the heated air expands, it is allowed to evacuate the vacuum vessel 5 by means of a one-way check valve 1. This one-way check valve 1 will close tight and maintain a vacuum inside the vacuum vessel 5 upon the natural cooling of the air inside the device and thus create a suction that holds the permeable medicated membrane 6 and the device in place, and at the same time increases blood flow under the treated area, thus facilitating a better transfer of medication from the Permeable medicated membrane 6 to the user tissue. One other important feature of this device is that it reduces the bacterial load under the auto generating vacuum bandage 14 area of treatment, which has a disinfecting effect to the tissue being treated. The vacuum vessel 5 herein can be of any shape and size, but in this illustration is shown in the square configuration embodiment.

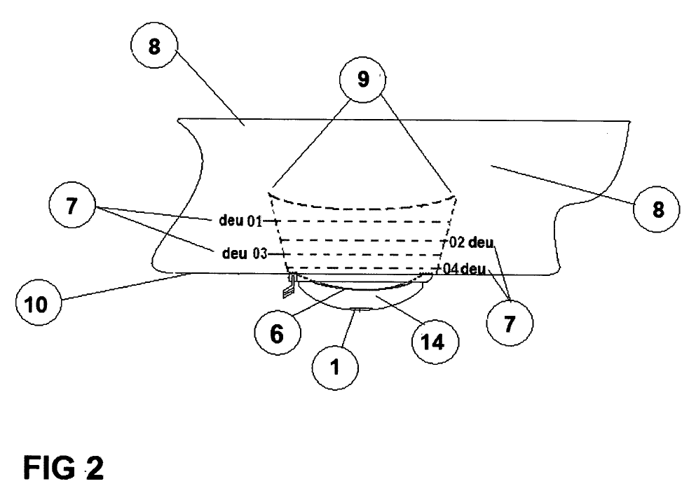

FIG. 2 is a graphical view of the decompressive energy gradient 9 to illustrate the pattern of deliverable decompressive energy to the soft tissue 8 being treated. The energy being greatest on the application surface 10 and diminishing as it penetrates the depths of the tissue 8. The units of measure are referred to herein as DEU 7 (decompressive energy unit). This diagram of DEU 7 gradient 9 is a generalized visual representation of a snap shot in time. The gradient 9 is a dynamic event and the DEU's 7 will change depending on tissue 10 density, the level of decompressive energy in the auto generating vacuum bandage 14, application time and a plurality of additional factors.

What is claimed is:

1. An apparatus for delivery of pharmacological compositions for treatment of tissue wounds comprising:
a. a vessel having a first and second opening, said first opening having a sealing member at the periphery of said opening, said vessel to be placed adjacent a tissue wound to be treated;
b. a one-way valve located at said second opening of said vessel wherein said one-way valve allows evacuation of said vessel;
c. a dermal patch impregnated with a heat activated pharmacological composition positioned opposite said vessel opening;
d. a chemical based heat source, said heat source self-enclosed within said vessel and having a tab in communication with the exterior of said vessel, wherein actuation of said tab initiates release of heat from said chemical based heat source to warm air inside said vessel to activate said dermal patch and evacuating air from said vessel thereby creating a vacuum within said vessel to compress and seal said dermal patch against said tissue wound for sustained delivery of said activated pharmacological treatment to said tissue wound.

2. An apparatus according to claim 1 wherein creation of said vacuum compresses said vessel sealing member around said dermal patch and reduces bacteria inside said vessel thereby disinfecting said tissue wound.

3. An apparatus according to claim 2 wherein compression seals said vessel around said dermal patch reducing bacteria infiltration.

4. An apparatus according to claim 2 wherein vascular blood flow to said tissue wound increases efficaciousness of said pharmacological compositions.

5. An apparatus according to claim 3 wherein vascular blood flow to said tissue wound increases efficaciousness of said pharmacological compositions.

6. An apparatus for delivery of pharmacological compositions for treatment of tissue wounds comprising:
a. an auto-generating vacuum vessel wherein said vacuum vessel has a first and second opening, said first opening having a sealing member at the periphery of said opening, said vacuum vessel to be placed adjacent a tissue wound to be treated;
b. a dermal patch impregnated with a heat activated pharmacological composition positioned opposite said first vacuum vessel opening and adjacent said tissue wound to be treated;
c. a one-way valve located at said second opening of said vessel wherein said one-way valve allows evacuation of said vacuum vessel;
d. a tab means in communication with the exterior of said vacuum vessel, wherein actuation of said tab initiates auto-generation of vacuum inside said vacuum vessel to warm air inside said vessel to activate said dermal patch and evacuate air from said vessel thereby creating a vacuum within said vessel to compress and seal said dermal patch against said tissue wound for sustained delivery of said activated pharmacological treatment to said tissue wound.

7. An apparatus according to claim 6 wherein creation of said vacuum compresses said vessel sealing member around said dermal patch and reduces bacteria inside said vessel thereby disinfecting said tissue wound.

8. An apparatus according to claim 7 wherein compression seals said vessel around said dermal patch reducing bacteria infiltration.

9. An apparatus according to claim 7 wherein auto-generation of vacuum increases vascular blood flow to said tissue in close proximity to said wound thereby increasing efficacy of said pharmacological compositions.
